(19) United States
(12) Patent Application Publication
(10) Pub. No.: US 2018/0184963 A1
(43) Pub. Date: Jul. 5, 2018

(54) SYSTEM AND METHOD FOR IMPROVING EMOTIONAL WELL-BEING BY VAGAL NERVE STIMULATION

(71) Applicant: BEYOND VERBAL COMMUNICATION LTD, Tel-Aviv (IL)

(72) Inventor: Yoram LEVANON, Ramat Hasharon (IL)

(73) Assignee: Beyond Verbal Communication Ltd, Tel-Aviv (IL)

(21) Appl. No.: 15/740,039

(22) PCT Filed: May 8, 2016
(86) PCT No.: PCT/IL2016/050483

§ 371 Date: Dec. 27, 2017

Related U.S. Application Data
(60) Provisional application No. 62/163,404, filed on May 19, 2015.

(51) Int. Cl. A61B 5/16; A61B 5/00; A61N 1/36; G10L 25/63

(52) U.S. Cl. CPC A61B 5/165; A61B 5/4803; A61N 1/36053; A61N 1/0558; G10L 25/63; A61N 1/36078; A61B 5/4836

(57) ABSTRACT

A system and methods for improving emotional well-being linked to physiological change in the human voice box by stimulating vagus nerve connected to motor and sensory functions in the voice box. The system and method further comprising providing a computer based system to sense metadata concerning a patient's emotional activity based on voice analysis; using said system, obtaining metadata concerning said patient during said activity undertaken; analyzing said data; and from said analysis, diagnosing the patient's emotional activity and providing a signal generator in a patient's body, said signal generator coupled to a first and a second electrode, said first electrode and second electrode coupled to a vagus nerve of the patient, wherein said first electrode is coupled to a main trunk of a vagus nerve of the patient, and the second electrode is coupled to a larynx branch of a vagus nerve of the patient; to, upon receiving at least one body data stream, provide stimulation pulses to the vagus nerve via said at least one electrode to elicit emotional treatment plan associated with said patient's emotional activity; and treat emotional well-being of said patient.

### FIELD OF THE INVENTION

The present invention relates to neural tissue stimulation techniques, and more particularly relates to techniques for improving emotional well-being by stimulating vagus nerve connected to motor and sensory functions in the voice box.

### BACKGROUND OF THE INVENTION

Emotional well-being is a term that links physical health to the emotional state of an individual.

Mental and emotional well-being is essential to overall health. On the positive side, enhanced emotional well-being is seen to contribute to upward spirals in increasing coping ability, self-esteem, performance and productivity at work, meaningful contributions to ones community, and even longevity. Early childhood experiences have lasting, measurable consequences later in life; therefore, fostering emotional well-being from the earliest stages of life helps build a foundation for overall health and well-being. Anxiety, mood (e.g., depression) and impulse control disorders are associated with a higher probability of risk behaviors (e.g., tobacco, alcohol and other drug use, risky sexual behavior), intimate partner and family violence, many other chronic and acute conditions (e.g., obesity, diabetes, cardiovascular disease, HIV/STIs), and premature death. Several systems for assessing and improving well-being have been presented. For example, EP patent No. 251,576 discloses a system and method for reducing the effects of negative emotional states by performing physiological measurements of a user with wearable. In another example, U.S. Pat. No. 6,878,111 discloses a system for measuring subjective well-being by receiving data or input that reflects an individual's subjective well-being and creating trends with a correlation module adapted to correlate and compare subjective and/or objective data. None of the cited patents above appear to disclose a system for configuring collective emotional well-being of an individual by evaluating manifestations of physiological change in the human voice box.

The vagus nerve is one of 12 pairs of cranial nerves that originate in the brain and is part of the autonomic nervous system, which controls involuntary body functions. The nerve passes through the neck as it travels between the chest and abdomen and the lower part of the brain. The nerve supplies nerve fibers to the pharynx (throat), trachea (windpipe), lungs, heart, esophagus, intestinal tract, as far as the transverse portion of the colon, and namely larynx (voice box). The vagus nerve also brings sensory information back to the brain from the ear, tongue, pharynx, and larynx.

Vagus nerve stimulation is a procedure that stimulates the vagus nerve with electrical impulses. The use of electrical stimulation for treatment of medical conditions is well known. For example, electrical stimulation of the brain with implanted electrodes has been approved for use in the treatment of various conditions, including pain and movement disorders such as essential tremor and Parkinson's disease. Another application of electrical stimulation of nerves is the treatment of radiating pain in the lower extremities by stimulating the sacral nerve roots at the bottom of the spinal cord [Paul F. WHITE, Shitong Li and Jen W. Chiu. Electroanalgesia: Its Role in Acute and Chronic Pain Management. Anesth Analg 92 (2001):505-513; U.S. Pat. No. 6,871,099 entitled Fully implantable microstimulator for spinal cord stimulation as a therapy for chronic pain, to Whitehurst, et al]. With vagus nerve stimulation, a device is surgically implanted under the skin on your chest. A wire is threaded under patient's skin connecting the device to the left vagus nerve. When activated, the device sends electrical signals along the vagus nerve to patient's brainstem, which then sends signals to certain areas in patient's brain. A number of vagus nerve stimulation techniques are known to treat different health conditions. Vagus nerve stimulation can be used to treat epilepsy when other treatments haven't worked. Vagus nerve stimulation is also a treatment for depression, and it's being studied for conditions such as multiple sclerosis, migraine and Alzheimer's disease. U.S. Pat. No. 8,634,922 discloses a system using electrical stimulation of the vagus nerve to treat epilepsy with minimized or no effect on the heart. Many therapeutic applications of electrical stimulation involve the surgical implantation of electrodes within a patient, for example U.S. Pat. No. 4,702,254 entitled Neurocybernetic prosthesis, to ZABARA; U.S. Pat. No. 6,341,236 entitled Vagal nerve stimulation techniques for treatment of epileptic seizures, to OSORIO et al; U.S. Pat. No. 5,299,569 entitled Treatment of neuropsychiatric disorders by nerve stimulation, to WERNICKE et al; G. C. ALBERT, C. M. Cook, F. S. Prato, A. W. Thomas. Deep brain stimulation, vagal nerve stimulation and transcranial stimulation: An overview of stimulation parameters and neurotransmitter release. Neuroscience and Biobehavioral Reviews 33 (2009) 1042-1060; GROVES D A, Brown V. J. Vagal nerve stimulation: a review of its applications and potential mechanisms that mediate its clinical effects. Neurosci Biobehav Rev 29 (2005):493-500; Reese TERRY, Jr. Vagus nerve stimulation: a proven therapy for treatment of epilepsy strives to improve efficacy and expand applications. Conf Proc IEEE Eng Med Biol Soc. 2009; 2009:4631-4634; Timothy B. MAPSTONE. Vagus nerve stimulation: current concepts. Neurosurg Focus 25 (3,2008):E9, pp. 1-4; ANDREWS, R. J. Neuromodulation. I. Techniques-deep brain stimulation, vagus nerve stimulation, and transcranial magnetic stimulation. Ann. N.Y. Acad. Sci. 993 (2003): 1-13; LABINER, D. M., Ahern, G. L. Vagus nerve stimulation therapy in depression and epilepsy: therapeutic parameter settings. Acta. Neurol. Scand. 115 (2007): 23-33. None of the cited above patents appear to apply neural tissue stimulation techniques for improving emotional well-being by stimulating vagus nerve connected to motor and sensory functions in the voice box.

Moreover, emotional well-being can be improved by reducing stress associated with the onset of mood and anxiety disorders including post-traumatic stress disorder (PTSD) as well as exacerbation of symptoms of these disorders and by increasing a patient's physiological resistance or resilience to stress. Beyond psychiatric disorders, stress can impact the course of diseases in individuals, such as inflammatory bowel and other immunological diseases.

In light of the above, there is a long term unmet need to provide system and methods for improving emotional well-being linked to physiological change in the human voice box by stimulating vagus nerve connected to motor and sensory functions in the voice box.

### SUMMARY OF THE INVENTION

It is hence one object of this invention to disclose a system and methods for improving emotional well-being linked to physiological change in the human voice box by stimulating vagus nerve connected to motor and sensory functions in the voice box.

It is yet another object of this invention to disclose a method for improving emotional well-being by stimulating vagus nerve, said method comprising the steps of: providing a computer based system to sense metadata concerning a patient's emotional activity based on voice analysis; using said system, obtaining metadata concerning said patient during said activity undertaken; analyzing said data; and from said analysis, diagnosing the patient's emotional activity and providing a signal generator in a patient's body, said signal generator coupled to a first and a second electrode, said first electrode and second electrode coupled to a vagus nerve of the patient, said first electrode is coupled to a main trunk of a vagus nerve of the patient, and the second electrode is coupled to a larynx branch of a vagus nerve of the patient; upon receiving at least one body data stream, said method further comprises a step of operating said signal generator to provide stimulation pulses to the vagus nerve via said at least one electrode to elicit emotional treatment plan associated with said patient's emotional activity; and emotional well-being of said patient is treated.

It is yet another object of this invention to disclose a system for improving emotional well-being by stimulating vagus nerve, said system comprising: a sensing module including a recording device adapted to sense at least one voice point of a patient and determine patient's emotional activity, said recording device having an output; an implantable signal generator providing stimulation energy; a first electrode and a second electrode coupled to a vagus nerve of the patient, wherein the first electrode is proximal to the brain relative to the second electrode, and the second electrode is coupled to a larynx branch of the vagus nerve; a microprocessor configured to said sensor signal for regulating said stimulation; a computer-readable medium including instructions for determining, using said recording device output, information concerning said patient's emotional activity; upon receiving at least one body data stream, said signal generator provides stimulation pulses to the vagus nerve via said at least one electrode to elicit emotional treatment plan associated with said patient's emotional activity; and emotional well-being of said patient is treated.

### BRIEF DESCRIPTION OF THE FIGURES

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

FIG. 1 schematically presents a system according to the present invention;

FIG. 2 is a flow diagram illustrating a method for improving emotional well-being linked to physiological change in the human voice box by stimulating vagus nerve connected to motor and sensory functions in the voice box.

### DETAILED DESCRIPTION OF THE INVENTION

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

As used herein, a “patient” is preferably a mammal, more preferably a human patient but can also be a companion animal (e.g., dog or cat), a farm animal (e.g., horse, cow, or sheep) or a laboratory animal (e.g., rat, mouse, or guinea pig). Preferable, the patient is human.

As used herein, the term “vagus nerve” is used in its broadest sense, and includes any nerves that branch off from the main vagus nerve, as well as ganglions or postganglionic neurons that are connected to the vagus nerve. The vagus nerve is also known in the art as the parasympathetic nervous system and its branches, and the cholinergic nerve. The vagus nerve enervates principal organs including, the pharynx, the larynx, the esophagus, the heart, the lungs, the stomach, the pancreas, the spleen, the kidneys, the adrenal glands, the small and large intestine, the colon, and the liver. Stimulation can be accomplished by direct stimulation of the vagus nerve or an organ served by the vagus nerve.

As used herein, “emotional well-being” refers to the one's state which characterized by the lack of the physiological implications which can be related to mental health concerns such as stress, depression, and anxiety. These in turn can contribute to physical ill-health such as digestive disorders, sleep disturbances, and general lack of energy.

As used herein, “stimulation” of the vagus nerve means activating or stimulating the vagus nerve by non-pharmacological means such as electrical, mechanical (e.g., vibration), heat or UV irradiation. Activation can be accomplished by direct stimulation of the vagus nerve or an organ served by the vagus nerve. The vagus nerve enervates principal organs including, the pharynx, the larynx, the esophagus, the heart, the lungs, the stomach, the pancreas, the spleen, the kidneys, the adrenal glands, the small and large intestine, the colon, and the liver.

As used herein, “treatment” includes prophylactic and therapeutic treatment. “Prophylactic treatment” refers to treatment before onset of an physocological condition to prevent, inhibit or reduce its occurrence. Therapeutic treatment is treatment of a subject who is already experiencing a physocological disorder.

The vagus nerve can be stimulated by numerous methods including manually, mechanically, electrically or by electromagnetic radiation. Mechanical means of nerve stimulation include stimulation by needle (e.g., acupuncture). There is evidence that response to acupuncture may be at least partially mediated by the vagus nerve. For example, it has been shown that the response to electroacupuncture is attenuated after vagotomy (Noguchi et al, Jpn. J. Physiol. 46(1): 53-58 (1996)). Mechanical stimulation may also include nerve stimulation using ultrasound as described, for example in Norton, BioMedical Engineering 2(1): 6 (2003). Stimulation of the vagus nerve using electromagnetic radiation includes applying infrared, visible or ultraviolet, heat or other energy source. The vagus nerve may also be stimulated by magnetic stimulation; a description of magnetic nerve stimulation is provided in Hsu et al, IEEE Trans Biomed Eng 50(11): 1276-85 (2003).

Examples of suitable vagus nerve stimulators are described, for example, in U.S. Pat. Nos. 4,702,254; 5,154,172; 5,231,988; 5,330,507; 6,473,644; 6,721,603; 6,735,471; U.S. Pat. App. Pub. 2004/0193231; and U.S. Pat. App. Pub. 2006/0178703. The teachings of all of these publications are incorporated herein by reference in their entirety.

According to one embodiment of the present invention, the vagus nerve is stimulated by delivering an electrical signal generated by any suitable vagus nerve stimulators. For example, a commercial vagus nerve stimulator or an electric probe can be used. The vagus nerve can be stimulated by means of either an implanted device or a device worn external to the patient's body, as described in U.S. Pat. No. 5,231,988 or as described in U.S. Pat. No. 5,330,507. Both patents describe apparati for stimulating the right or left vagus nerve with continuous and/or phasic electrical signal.

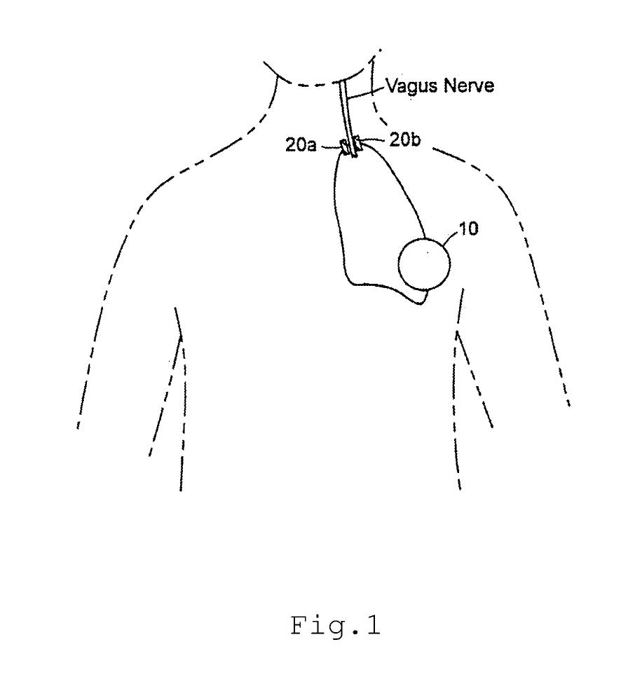

The principles, systems and methods for determining the emotional subtext of a spoken utterance used in this invention are those disclosed by Levanon et al. in PCT Application WO 2007/072485; a detailed description of their method of intonation analysis may be found in that source. Reference is made to FIG. 1, presenting a schematic and generalized presentation of the present invention where signal generator 10 is implanted in the patient's chest in a pocket formed by the surgeon just below the skin. One suitable location for the generator is in the patient's chest, as a pacemaker pulse generator would be implanted, with the electrodes 20 a and 20 b imlanted in the patient's neck. Electrodes 20 a and 20 b can be bipolar stimulating electrodes of the type described in U.S. Pat. No. 4,573,481, incorporated herein by reference in its entirety. In this embodiment, electrodes form an assembly which is surgically implanted on the vagus nerve in the patient's neck. The two electrodes are wrapped around the vagus nerve, and the assembly is secured to the nerve by a spiral anchoring tether as disclosed in U.S. Pat. No. 4,979,511 and in U.S. Pat. App. 2006/0178703, incorporated herein by reference in its entirety. Structurally, the electrode assembly can comprise two ribbons of platinum which are individually bonded to each of the two spiral loops wrapped around the vagus nerve. Each loop further includes silicone rubber. An additional helical loop that includes silicon rubber is provided to tether the electrode assembly to the vagus nerve. The inner diameter of the helical bipolar electrodes may typically be about two millimeters (mm), and an individual spiral is about seven mm long (measured along the axis of the nerve). Instead of implanting the electrode assembly in the patient's neck, the assembly may be implanted on the vagus nerve as it enervated any of the organs listed above. The implantation of electrodes 20 a and 20 b is accomplished in substantially the same manner as was described for the neck location.

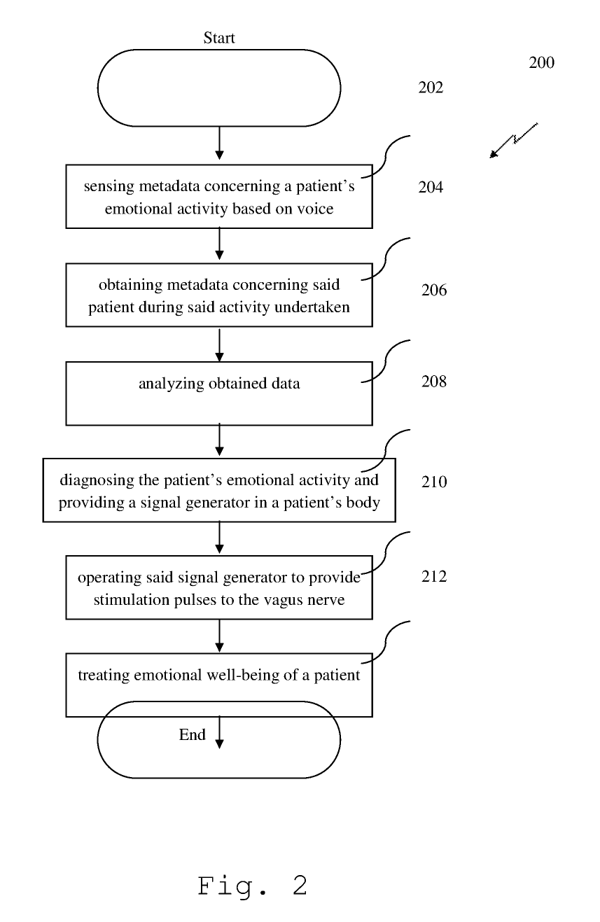

Reference is now made to FIG. 2, presenting a flow diagram illustrating a method for improving emotional well-being linked to physiological change in the human voice box by stimulating vagus nerve connected to motor and sensory functions in the voice box. Said method comprises, for a predetermined number of repetitions 20, steps of providing a computer based system to sense metadata concerning a patient's emotional activity based on voice analysis 202; using said system, obtaining metadata concerning said patient during said activity undertaken 204; analyzing said data 206; and from said analysis, diagnosing the patient's emotional activity and providing a signal generator in a patient's body, said signal generator coupled to a first and a second electrode, said first electrode and second electrode coupled to a vagus nerve of the patient, wherein said first electrode is coupled to a main trunk of a vagus nerve of the patient, and the second electrode is coupled to a larynx branch of a vagus nerve of the patient 208; operating said signal generator to provide stimulation pulses to the vagus nerve via said at least one electrode to elicit emotional treatment plan associated with said patient's emotional activity 210; and treating emotional well-being of said patient 212.

What is claimed is:

1. A method for improving emotional well-being by stimulating a vagus nerve, said method comprising the steps of:
a. providing a computer based system to sense metadata concerning a patient's emotional activity based on voice analysis;
b. using said system, obtaining metadata concerning said patient during said activity undertaken;
c. analyzing said data; and
d. from said analysis, diagnosing the patient's emotional activity and providing a signal generator in said patient's body, said signal generator coupled to a first and a second electrode, said first electrode and second electrode coupled to a vagus nerve of the patient, wherein said first electrode is coupled to a main trunk of said vagus nerve of the patient, and the second electrode is coupled to a larynx branch of said vagus nerve of the patient;
wherein, upon receiving at least one body data stream, said method further comprises a step of operating said signal generator to provide stimulation pulses to the vagus nerve via said at least one electrode to elicit emotional treatment plan associated with said patient's emotional activity;
wherein emotional well-being of said patient is treated automatically; and
wherein at least a portion of said metadata concerns determining a pattern and fluctuation in said patient's voice.

2. The method according to claim 1, wherein said emotional activity is a normal activity of the person.

3. The method according to claim 1 further including the step of providing, based on said analysis, vagus nerve stimulation to prevent the occurrence of a negative emotional activity, and/or to mitigate the effects thereof.

4. The method according to claim 1 wherein vagus nerve stimuli is further provided to elicit possible responses characteristic of patient's negative emotional activity.

5. The method according to claim 4 wherein said patient undergoing physical and/or mental health related treatment plan.

6. (canceled)

7. (canceled)

8. The method according to claim 1 further including the step of recording said data for future analysis.

9. The method according to claim 1 further including the steps of transmitting said data for a remote location for analysis and visual or audio information back to the patient while they are engaged in said activity.

10. The method according to claim 1 wherein vagus nerve stimuli is further provided to reduce a stress-related disorder or symptoms in said patient by increasing said patient's physiological resistance or resilience to stress.

11. The method according to claim 10 wherein vagus nerve stimuli is further provided to reduce a risk of stress-related inflammatory bowel disorders and immunological conditions.

12. The method according to claim 11, wherein said inflammatory disorder is selected from the group consisting of appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome, Hodgkin's disease, sepsis, endotoxic shock, allograft rejection, rheumatoid arthritis, adult respiratory distress syndrome, asthma, systemic lupus erythematosis, pancreatitis, peritonitis, burns, myocardial ischemia, allograft rejection, graft-versus-host disease, congestive heart failure, organ ischemia, reperfusion injury, cachexiak cystic fibrosis, appendicitis, ulcerative colitis, Crohn's disease, allergy, reperfusion injury, systemic lupus erythematosus, hepatitis, Behcet's syndrome, multiple sclerosis and atherosclerosis.

13.-14. (canceled)

15. A system for improving emotional well-being by stimulating vagus nerve, said system comprising:
a. a sensing module including a recording device adapted to sense at least one voice point of a patient and determine patient's emotional activity, said recording device having an output;
b. an implantable signal generator providing stimulation energy;
c. a first electrode and a second electrode coupled to a vagus nerve of the patient, wherein the first electrode is proximal to the brain relative to the second electrode, and the second electrode is coupled to a larynx branch of the vagus nerve;
d. a microprocessor configured to said sensor signal for regulating said stimulation;
e. a computer-readable medium including instructions for determining, using said recording device output, information concerning said patient's emotional activity;
wherein, upon receiving at least one body data stream, said signal generator provides stimulation pulses, regulated by said microprocessor, to the vagus nerve via said at least one electrode to elicit an emotional treatment plan associated with said patient's emotional activity, said emotional activity determined by instructions included in said computer readable medium;
wherein emotional well-being of said patient is treated automatically; and
wherein at least a portion of said metadata concerns determining a pattern and fluctuation in said patient's voice.

16. The system according to claim 15, wherein said emotional activity is a normal activity of the person.

17. The system according to claim 15, wherein, based on said analysis, vagus nerve stimulation by said signal generator prevents the occurrence of a negative emotional activity, and/or mitigates the effects thereof.

18. The system according to claim 15, wherein vagus nerve stimuli is further provided to elicit possible responses characteristic of patient's negative emotional activity.

19. The system according to claim 15, wherein said patient undergoes a physical and/or mental health related treatment plan.

20. (canceled)

21. (canceled)

22. The system according to claim 15, wherein said recorded by a recording device data is used for future analysis.

23. The system according to claim 15, wherein said computer-readable medium including instructions for transmitting said data for a remote location for analysis and visual or audio information back to the patient while they are engaged in said activity.

24. The system according to claim 15, wherein vagus nerve stimuli is further provided to reduce a stress-related disorder or symptoms in said patient by increasing said patient's physiological resistance or resilience to stress.

25. The system according to claim 21 wherein vagus nerve stimuli is further provided to reduce a risk of stress-related inflammatory bowel disorders and immunological conditions.

26. The system according to claim 25, wherein said inflammatory disorder is selected from the group consisting of appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome, Hodgkin's disease, sepsis, endotoxic shock, allograft rejection, rheumatoid arthritis, adult respiratory distress syndrome, asthma, systemic lupus erythematosis, pancreatitis, peritonitis, burns, myocardial ischemia, allograft rejection, graft-versus-host disease, congestive heart failure, organ ischemia, reperfusion injury, cachexia, cystic fibrosis, appendicitis, ulcerative colitis, Crohn's disease, allergy, reperfusion injury, systemic lupus erythematosus, hepatitis, Behcet's syndrome, multiple sclerosis and atherosclerosis.

27.-28. (canceled)
